(19) United States
(12) Patent Application Publication
(10) Pub. No.: US 2003/0114732 A1
(43) Pub. Date: Jun. 19, 2003

(54) Sheath for guiding imaging instruments

(75) Inventors: William E. Webler, Escondido, CA (US); Gary Schneiderman, San Ramon, CA (US); Douglas J. Seiffert, Pembroke, FL (US)

Correspondence Address: COUDERT BROTHERS LLP, 600 Beach Street, 3rd Floor, San Francisco, CA, 94109-1314, US

(73) Assignee: Advanced Cardiovascular Systems, Inc.

(21) Appl. No.: 10/025,149

(22) Filed: Dec. 18, 2001

(51) Int. Cl. A61B 1/00

(52) U.S. Cl. 600/121

(57) ABSTRACT

The invention is directed to apparatus, methods and systems including a sheath for use with intracorporeal optical imaging instruments such as imaging guidewires, catheters, or endoscopes. The invention provides a sheath suitable for guiding an enclosed instrument, that is effective to guide the placement within a patient's body and replacement to a distal position after retraction of the imaging instrument, as during an imaging scan. The sheaths may include at least a portion that is translucent to a desired wavelength of radiation. The translucent portion may have an index of refraction similar to the index of refraction of a bodily fluid such as blood plasma, or an artificial fluid suitable for introduction into a body lumen.

### FIELD OF THE INVENTION

The present invention relates generally to intracorporeal methods and apparatus for positioning imaging instruments within a body lumen. In particular, the invention is directed to a sheath for guiding the placement of optical imaging instruments such as imaging guidewires, catheters, or endoscopes, and methods for using such sheaths.

### BACKGROUND OF THE INVENTION

Many intracorporeal clinical procedures require the insertion of wires, tubes, probes or other objects into a body lumen of a patient. For example, guidewires and catheters may be used for gaining access to the coronary vasculature, as in an angiogram or in angioplasty. A guidewire is a thin, flexible device used to provide a guiding rail to a desired location within the vasculature (or other body cavity) of a patient. A balloon catheter is a device with an interior lumen with at least a portion of the catheter being able to expand. In coronary angioplasty, a balloon catheter, guided by a guidewire, is positioned within a partially-occluded coronary artery where its balloon portion is expanded in order to press against and enlarge the lumen of a blood vessel in which it is situated. Alternatively, endoscopy requires the introduction of an endoscope into a lumen of a patient, as may be done during a colonoscopy.

Imaging of internal body lumens provides clinicians with information useful in many clinical situations and procedures. Imaging may be accomplished using electromagnetic radiation (such as, e.g., optical radiation, infrared radiation, and radiofrequency radiation). For example, where a patient is suspected of having an occlusion in an artery, optical imaging of the artery and the artery wall can provide information about the type, severity and extent of an occlusion or lesion and so improve the diagnosis and treatment of the patient. Intracorporeal imaging is useful for the placement of guidewires, catheters, endoscopes, and other instruments in desired locations within a patient's body, typically within a body lumen. The ability to decide where to locate a catheter during a clinical procedure can be improved by providing interior images of the body lumen, such as the blood vessels during angioplasty or the colon during colonoscopy. It is often critical to the success of an angioplasty procedure that a balloon catheter be properly located within a blood vessel. Thus imaging by guidewire, catheter, endoscope or other such device can be of great importance to the success of a clinical procedure. However, radiation passing from one medium to another may be refracted; an image derived from refracted radiation may be distorted, and such distortion may compromise the success of a clinical procedure.

Optical imaging endoscopes, guidewires and catheters have been described, as in U.S. Pat. Nos. 5,321,501 and 5,459,570 to Swanson et al., and U.S. Pat. No. 6,134,003 to Tearney et al. Catheters configured for optical imaging using non-visible light may be useful as well, as disclosed in U.S. Pat. No. 5,935,075 to Cassells et al. Such imaging devices typically use an optical fiber to carry light. All patents, supra and infra, are hereby incorporated by reference in their entirety.

In coronary angioplasty, a guidewire and an angioplasty catheter are threaded through a patient's vascular bed to bring the distal ends of the guidewire and catheter to and beyond the site of the lesion. For effective use of a balloon angioplasty catheter, the distal end of the balloon angioplasty catheter preferably extends to a position distal to the lesion. For this reason, it is vital that the clinician have accurate knowledge of the extent of the lesion.

To do so, the imaging instrument must be located within the body lumen containing the lesion, positioned adjacent or near to the lesion. Typically, an imaging instrument will be advanced distally into the lumen, until a lesion is encountered. The instrument will often be advanced further distally to determine the extent and margins of the lesion, and to position therapeutic instruments across the lesion so that the entire lesion may be treated.

After such distal positioning within a lumen across a lesion, where a clinician wishes to observe or document the condition of a body lumen during an invasive procedure, an imaging instrument may be retracted proximally in order to scan the lumen to obtain imaging information pertaining to the lesion. As a result of the retraction for scanning purposes, the instrument's position across the lesion in the vessel may be lost. Losing the position is potentially traumatic, and it is sure to be time-consuming and laborious to regain the lost position. This loss makes it impractical and undesirable to use the “pull back” mode of imaging, which is otherwise to be preferred. Accordingly, devices and methods for reducing distortion of intracorporeal images, and for readily returning an imaging instrument to a desired location after retraction are desired.

### SUMMARY OF THE INVENTION

The invention provides sheaths and methods of using sheaths for guiding intracorporeal imaging instruments to a desired location within a body lumen. The sheaths provide a guiding path for an enclosed imaging instrument allowing the instrument's repositioning at a distal location within a body lumen after retraction to a more proximal location. In particular, the invention involves using sheaths with intracorporeal imaging instruments such as imaging guidewires, catheters, or endoscopes, and methods for using such sheaths. The intracorporeal imaging instrument may be an ultrasound instrument, or an instrument sensitive to electromagnetic radiation, such as an optical instrument. At least a portion of the sheath may be configured with an index of refraction closely matching that of the surrounding medium.

An embodiment of the invention provides a sheath configured to enclose and to engage an intracorporeal imaging instrument, allowing the imaging instrument to travel longitudinally within the sheath. The sheath is configured to allow enclosed instruments to easily slide and travel within the sheath. In some embodiments of the invention, the sheath has a smooth inner surface. In further embodiments, the sheath is made of materials providing smooth surfaces that present little resistance to movement of objects within the sheath. Image information may be acquired by the imaging instrument during longitudinal movement, for example in a proximal direction (“pullback”), the sheath providing a path and acting as a guide to return the instrument to a previous distal position occupied before the image acquisition. Imaging from within a sheath provides a smooth consistent path for the imaging instrument to follow during longitudinal movement, and prevents rapid changes in the instrument's position within the lumen during pullback.

A sheath embodying features of the invention may be translucent or transparent to a wavelength of radiation for guiding an intracorporeal imaging instrument sensitive to radiation of that wavelength. In some embodiments of the invention, the radiation is optical radiation, the wavelength is an optical radiation wavelength, and the imaging instrument is an intracorporeal optical imaging instrument. Thus, in some embodiments of the invention, a distal portion of the sheath may be translucent to that wavelength of electromagnetic radiation, such as optical radiation. In certain embodiments of the system, the sheath is configured to minimize refraction by including a material with an index of refraction similar to the index of refraction of a bodily fluid or of an artificial bodily fluid. Thus, the sheath may include a material or materials with an index of refraction of between about 1.2 and about 1.5, or, in further embodiments of the invention, between about 1.3 and about 1.4, particularly about 1.34. In some embodiments of the invention, the sheath for guiding an intracorporeal optical imaging instrument includes a material selected from the group consisting of fluorinated ethylene propylene (FEP), polytetrafluorethylene (Teflon®), perfluoroalkoxy polymers (PFA), ethylene tetrafluoroethylene copolymers (ETFE), and blends thereof. The sheath may also have a radiopaque marker or markers. In some embodiments of the invention, the sheath may be configured with an over-the-wire design or with a rapid exchange design and may be configured to receive a guidewire.

The invention provides systems for intracorporeal imaging, including a sheath and an intracorporeal imaging instrument. The invention also provides an intracorporeal imaging system including an intracorporeal imaging instrument sensitive to a wavelength of optical radiation, and a sheath comprising a material translucent to that wavelength of optical radiation for guiding said intracorporeal imaging instrument. The sheath of the systems of the invention may be configured to minimize refraction by including a material or materials with an index of refraction of between about 1.2 and about 1.5, or between about 1.3 and about 1.4, particularly about 1.34.

The invention also provides a method for guiding an imaging instrument within a body lumen, including some or all of the steps of locating within a body lumen a sheath having a distal end and a proximal end, locating an intracorporeal imaging instrument within the sheath at a location within said body lumen, moving the intracorporeal imaging instrument within at least a portion of the sheath in a proximal direction, moving the intracorporeal optical imaging instrument within at least a portion of the sheath in a distal direction, and obtaining image information.

The sheaths and methods of the invention find use in clinical and diagnostic procedures such as imaging of an internal lumen of a patient's body. Imaging of an internal lumen of a patient's body typically includes insertion into a patient's body of an intracorporeal imaging device, such as an imaging guidewire, imaging catheter, endoscope, or other instrument, and movement of the imaging instrument within a region of interest within the patient's body. Images or optical information may be gathered from a moving instrument in order to obtain a scan or scanned image of an extended area within the body lumen. Scanned images are typically preferred to static images of a single location.

The sheaths and methods of the invention provide such advantages as providing a guide that enables clinicians to readily return an intracorporeal instrument to a prior location after retracting it from a distal position within a body lumen, providing high fidelity images from within a translucent sheath that minimizes distortion without requiring an imaging instrument to extend beyond the sheath during use, and providing guidance for the movement of intracorporeal instruments. Sheaths of the invention protect internal body surfaces while allowing placement and movement of intracorporeal instruments, providing a smooth surface to minimize motion artifacts and to minimize possible damage to a body lumen by intracorporeal instruments.

### BRIEF DESCRIPTION OF THE DRAWINGS

FIG. 1A is a partial perspective view with a partial longitudinal cross-section of a system embodying features of the invention including a translucent sheath enclosing an intracorporeal imaging instrument.

FIG. 1B is a transverse cross-sectional view of the system of FIG. 1A taken along line 1B-1B.

FIG. 1C is a transverse cross-sectional view of the system of FIG. 1A taken along line 1C-1C.

FIG. 2A is a partial perspective view with a partial longitudinal cross-sectional view of a sheath embodying features of the invention, the sheath enclosing an imaging instrument and a guidewire and having two lumens configured in a rapid exchange (RX) configuration.

FIG. 2B is a transverse cross-sectional view of the sheath of FIG. 2A taken along line 2B-2B.

FIG. 2C is a transverse cross-sectional view of the sheath of FIG. 2A taken along line 2C-2C.

FIG. 3A is a partial perspective view with a longitudinal cross-sectional view of a sheath, imaging instrument, and guidewire, the sheath having two lumens configured in an over-the-wire (OTW) configuration.

FIG. 3B is a transverse cross-sectional view of the sheath of FIG. 3A taken along line 3B-3B.

FIG. 3C is a transverse cross-sectional view of the sheath of FIG. 3A taken along line 3C-3C.

FIG. 4A is a partial perspective view with a longitudinal cross-sectional view of a sheath embodying features of the invention, having multiple lumens configured in a RX configuration and enclosing an imaging instrument, a guidewire and another intracorporeal instrument.

FIG. 4B is a partial perspective view with a longitudinal cross-sectional view of a sheath and instruments similar to those shown in 4A, the sheath having multiple lumens configured in an OTW configuration.

FIG. 4C is a transverse cross-sectional view of a multiple-lumen sheath as illustrated in FIGS. 4A and 4B taken along the line 4C-4C.

FIG. 5A is a partial perspective view with a partial longitudinal cross-sectional view of a sheath embodying features of the invention enclosing an imaging instrument stabilized within the sheath by a balloon, the guidewire lumen being in the RX configuration.

FIG. 5B is a partial perspective view with a partial longitudinal cross-sectional view of a sheath embodying features of the invention enclosing an imaging instrument stabilized within the sheath by a balloon, the guidewire lumen being in the OTW configuration.

FIG. 5C is a transverse cross-sectional view of a sheath embodying features of the invention having two lumens and enclosing an imaging instrument and a guidewire.

FIG. 5D is a partial perspective view with a partial longitudinal cross-sectional view of a sheath embodying features of the invention having two lumens enclosing an imaging instrument and a guidewire, showing the imaging instrument completely enclosed within a lumen.

FIG. 5E is a partial perspective view with a partial longitudinal cross-sectional view of a sheath embodying features of the invention having two lumens enclosing an imaging instrument and a guidewire, showing the imaging instrument extending distally out of a lumen.

FIG. 6A is a partial perspective view with a partial longitudinal cross-sectional view of a sheath embodying features of the invention having two lumens, enclosing a guidewire in a RX configuration and an imaging instrument in position within a body lumen prior to beginning an imaging scan.

FIG. 6B is a partial perspective view with a partial longitudinal cross-sectional view of a sheath embodying features of the invention having two lumens, enclosing a guidewire in a RX configuration and an imaging instrument after completion of an imaging scan.

FIG. 6C is a partial perspective view with a partial longitudinal cross-sectional view of a sheath embodying features of the invention having two lumens, enclosing a guidewire in a RX configuration and an imaging instrument following re-positioning of the imaging instrument at its prior location within the sheath and within the body lumen.

FIG. 6D is a partial perspective view with a partial longitudinal cross-sectional view of a sheath embodying features of the invention having two lumens, enclosing a guidewire in an OTW configuration and an imaging instrument in position within a body lumen prior to beginning an imaging scan.

FIG. 6E is a partial perspective view with a partial longitudinal cross-sectional view of a sheath embodying features of the invention having two lumens, enclosing a guidewire in an OTW configuration and an imaging instrument after completion of an imaging scan.

FIG. 6F is a partial perspective view with a partial longitudinal cross-sectional view of a sheath embodying features of the invention having two lumens, enclosing a guidewire in an OTW configuration and an imaging instrument following re-positioning of the imaging instrument at its prior location within the sheath and within the body lumen.

FIG. 7A is a transverse cross-sectional view of a sheath embodying features of the invention having two lumens and enclosing a guidewire and an imaging instrument in position within a body lumen prior to beginning an imaging scan.

FIG. 7B is a transverse cross-sectional view of a sheath embodying features of the invention having two lumens and enclosing a guidewire and an imaging instrument after completion of an imaging scan.

FIG. 7C is a transverse cross-sectional view of a sheath embodying features of the invention having two lumens and enclosing a guidewire and an imaging instrument following re-positioning of the imaging instrument at its prior location within the sheath and within the body lumen.

### DETAILED DESCRIPTION OF THE INVENTION

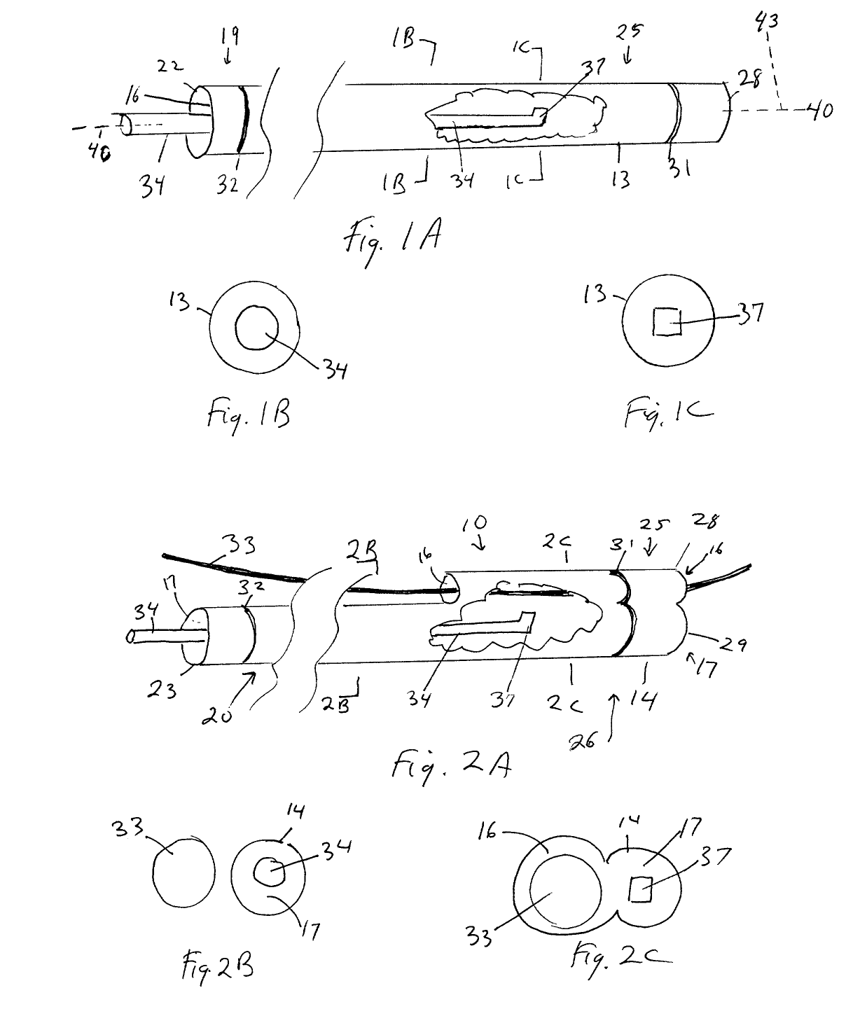

Shown in FIG. 1A is an intracorporeal imaging system 10 including a novel sheath 13 and an intracorporeal optical imaging instrument 34. The sheath 13 includes material translucent to a wavelength of radiation (such as, e.g., optical radiation of a wavelength between about 0.1 micron to about 3 micron), and is configured to define a lumen 16, with a proximal portion 19, a proximal aperture 22, a distal portion 25, a distal aperture 28. Sheath 13 has a distal radiopaque marker 31 situated at a position on the distal portion 25 to aid the determination of the position of distal portion 25 within a patient's body and a proximal marker 32, which typically remains outside a patient's body during a procedure, situated at a position on a proximal portion 19 to aid the determination of the position of the sheath. Sheath 13 is shown enclosing an imaging instrument 34 with distal imaging elements 37 sensitive to the wavelength of optical radiation. Portions of objects within the sheath 13, such as imaging instrument 34, are shown in the partial cross sections within scalloped lines in FIG. 1A and in subsequent figures. Instruments and methods for using instruments suitable for use with sheaths of the present invention are disclosed in co-owned applications Ser. No. XXX, “Optical Guidewire Having Windows or Apertures” to Jalisi et al., application Ser. No. YYY “Rotatable Ferrules and Interfaces for Use with an Optical Guidewire,” to Webler et al., and application Ser. No. ZZZ “Methods for Forming an Optical Window for an Intracorporeal Device and for Joining Parts,” to Webler et al., all of which are filed concurrently herewith, and the disclosures of which are all hereby incorporated by reference in their entirety.

Imaging instrument 34 is able to move freely along directions substantially along longitudinal axis 40, both distally and proximally, although the sheath 13 guides imaging instrument 34 so as to generally constrain movement in a lateral direction 43 generally perpendicular to longitudinal axis 40. FIG. 1B shows a transverse cross-section taken along line 1B-1B in FIG. 1A through imaging instrument 34, and FIG. 1C shows a transverse cross-section taken along line 1C-1C in FIG. 1A through imaging elements 37.

Sheaths embodying features of the invention include at least one lumen having an inner surface configured to allow ready movement of objects enclosed within the sheath. In some embodiments of the invention, the sheath inner surface is configured to slidingly engage an intracorporeal imaging instrument effective to guide the longitudinal movement of the intracorporeal imaging instrument within the lumen. For example, the sheath may be made using materials such as plastics, including polymers such as Teflon® (polytetrafluorethylene) and other polymers and polymer blends, that provide smooth slippery surfaces. Sheaths embodying features of the invention are typically translucent to radiation over a range of optical radiation wavelengths. Such optical radiation includes ultraviolet radiation, visible radiation, and infrared radiation, including, for example, radiation with wavelengths of between about 0.1 micron to about 2 micron in wavelength.

Commonly, procedures involving introduction of instruments into a cardiovascular lumen include insertion of a guidewire into the vasculature of a patient. The guidewire may be of any suitable kind. The guidewire may be useful for guiding an optical imaging instrument into position within a patient, or may be an imaging guidewire which itself is an optical imaging instrument. Guidewires and instruments for use with guidewires may be of the rapid-exchange (RX) type or of the over-the-wire (OTW) type. A rapid-exchange type instrument is configured to be mounted onto a guidewire by a short sleeve or loop typically located at the distal end of the instrument. An over-the-wire type instrument is configured to be mounted onto a guidewire by a sleeve or other attachment fixture that extends substantially along the entire length of the over-the-wire instrument. Sheath 13 illustrated in FIGS. 1A-1C is configured as an OTW type catheter. Alternatively, sheaths embodying features of the invention may have a RX configuration, which one skilled in the art will be able to implement based on the present disclosure.

As used herein, “translucent” means permitting the passage of radiation useful for imaging. A translucent material is a material that allows passage of radiation. A transparent material is a translucent material that produces little or no diffusion of radiation passing though it, so that images may be reliably formed from radiation passing through a transparent material. Thus, for example, radiation used for imaging passes through a translucent material with greater or lesser degrees of diffusion, and passes through a transparent material with little or no diffusion at all so that images may be reliably formed. An imaging instrument may use any suitable form of radiation to form an image, including ultrasound, electromagnetic radiation, and other forms of radiation. An optical imaging instrument is sensitive to a wavelength of optical radiation, such as ultraviolet radiation, visible light, infrared radiation, and radiofrequency radiation. Translucent materials suitable for use with optical imaging instruments include materials that allow passage of radiation of wavelengths between about 0.1 micron to about 3 micron, particularly radiation of wavelengths between about 0.75 micron to about 2.5 micron, or radiation of wavelengths between about 0.1 micron to about 1 micron.

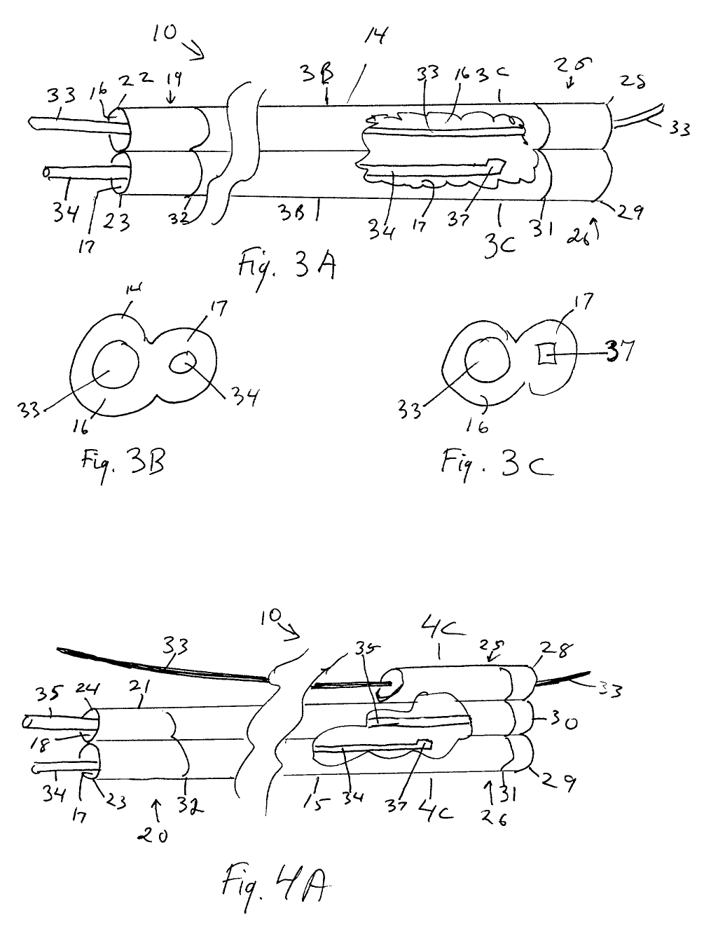

Shown in FIGS. 2A, 2B and 2C is an intracorporeal imaging system 10 including a sheath 14 embodying features of the invention made using a material translucent to a wavelength of optical radiation. A portion of the sheath and instruments enclosed within the sheath are shown in longitudinal cross-section in FIG. 2A. The sheath is configured so as to define two lumens, lumens 16 and 17, in a RX configuration. The sheath 14 includes a first lumen 16 having a proximal portion 19 with a proximal aperture 22 and a distal portion 25 having a distal aperture 28, and a second lumen 17 having a proximal portion 20 with a proximal aperture 23 and a distal portion 26 having a distal aperture 29. Radiopaque markings 31 on distal portions 25 and 26 are useful for identifying the position of the system within a patient's body, and a proximal marking 32 (or, alternatively, multiple markings 32) on proximal portion 20 is similarly useful as indicators of the position of the system. The sheath 14 is shown enclosing a guidewire 33 within lumen 16 and a fiber-optical imaging instrument 34 within lumen 17. Sheath 14 includes translucent material. Imaging elements 37 of the imaging instrument 34 are sensitive to the optical wavelength to which the material is translucent. Guidewire 33 and imaging instrument 34 are able to move freely in a direction generally parallel to longitudinal axis 40, although their movement in lateral direction 43 is generally constrained by sheath 14. The translucent sheath 14 illustrated in FIGS. 2A-2C is configured as a RX type catheter, so that guidewire 33 accesses sheath lumen 16 via proximal aperture 22 that is configured as a RX aperture in sheath 14.

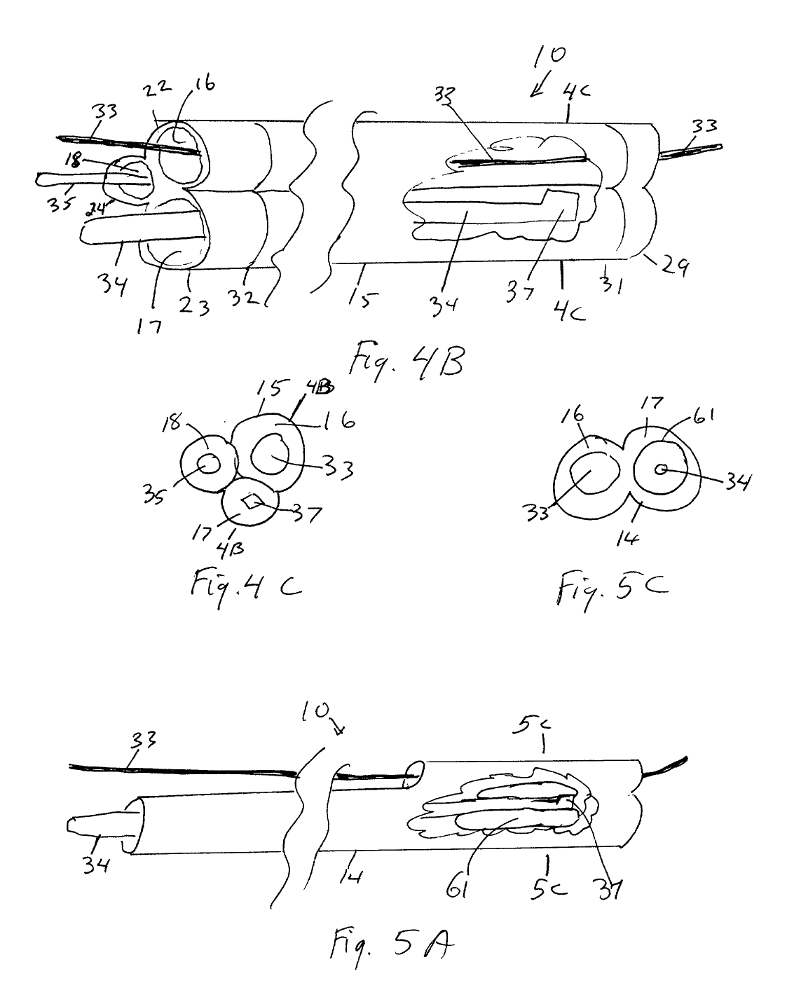

Similarly, an intracorporeal imaging system 10 shown in FIGS. 3A-3C includes a sheath 14 embodying features of the invention made using a material translucent to a wavelength of optical radiation. The sheath illustrated in FIGS. 3A-3C is configured so as to define two lumens, lumens 16 and 17, in an OTW configuration. The sheath 14 includes a first lumen 16 having a proximal portion 19 with a proximal aperture 22 and a distal portion 25 having a distal aperture 28, and a second lumen 17 having a proximal portion 20 with a proximal aperture 23 and a distal portion 26 having a distal aperture 29. FIG. 3A includes a portion shown in longitudinal cross-section. Radiopaque markings 31 on distal portions 25 and 26 are useful for identifying the position of the system within a patient's body, and proximal markings 32 on proximal portions 19 and 20 are similarly useful as indicators of the position of the system. The translucent sheath 14 illustrated in FIGS. 3A-3C includes an OTW catheter, so that guidewire 33 accesses sheath lumen 16 via proximal aperture 22 that is configured as an OTW aperture in sheath 14.

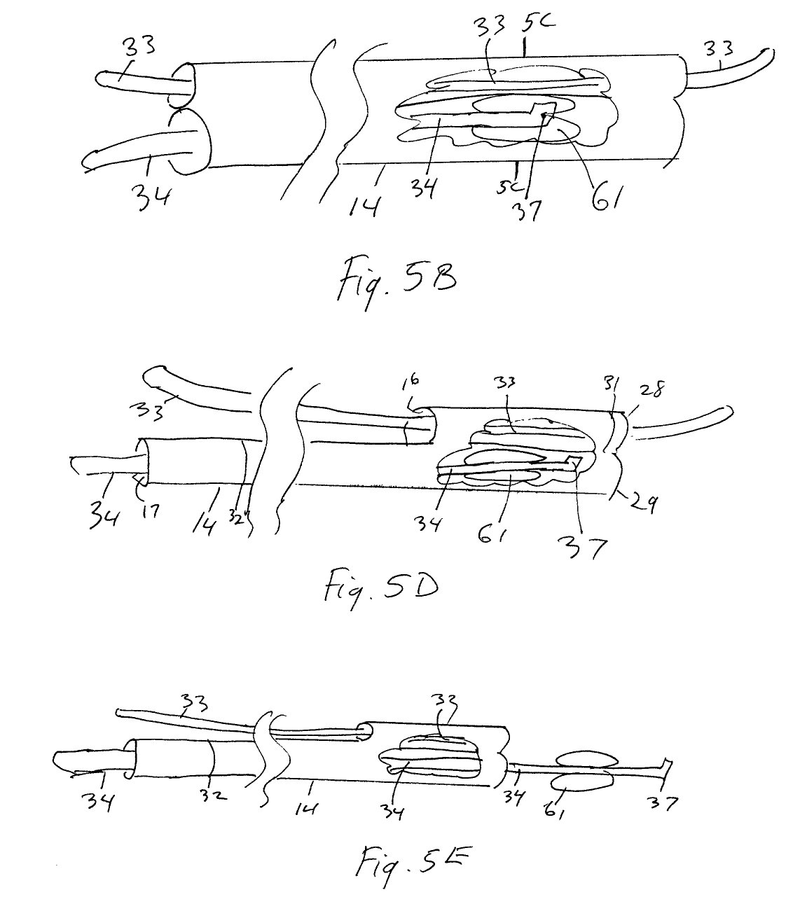

Shown in FIGS. 4A, 4B and 4C is an intracorporeal imaging system 10 including a sheath 15 configured to define three lumens, lumens 16, 17 and 18. The sheath 15 includes a first lumen 16 having a proximal portion 19 with a proximal aperture 22 and a distal portion 25 having a distal aperture 28, a second lumen 17 having a proximal portion 20 with a proximal aperture 23 and a distal portion 26 having a distal aperture 29, and a third lumen 18 having a proximal portion 21 with a proximal aperture 24 and a distal portion 27 having a distal aperture 30. Radiopaque markings 31 on distal portions 25, 26 and 27 are useful for identifying the position of the system within a patient's body, and proximal markings 32 on proximal portions 20 and 21 (FIG. 4A) or on proximal portions 19, 20 and 21 (FIG. 4B) are similarly useful as indicators of the position of the system. The sheath 15 is shown enclosing a guidewire 33 that is able to move freely in a direction generally parallel to longitudinal axis 40 within lumen 16, although its movement in lateral direction 43 is generally constrained by sheath 14. The translucent sheath 15 illustrated in FIG. 4A is configured as a RX catheter, so that guidewire 33 accesses sheath lumen 16 via proximal aperture 22 that is configured as a RX aperture. FIG. 4A includes a position shown in longitudinal cross-section. The translucent sheath 15 illustrated in FIGS. 4B includes an OTW catheter, so that guidewire 33 accesses sheath lumen 16 via proximal aperture 22 that is configured as an OTW aperture. FIG. 4B includes a partial longitudinal cross-section taken along line 4B-4B shown in FIG. 4C. A transverse cross-sectional view of a multi-lumen sheath is shown in FIG. 4C. Imaging elements 37 of the imaging instrument 34 within lumen 17 are sensitive to the optical wavelength to which the material is translucent. Third lumen 18 encloses an additional intracorporeal instrument 35.

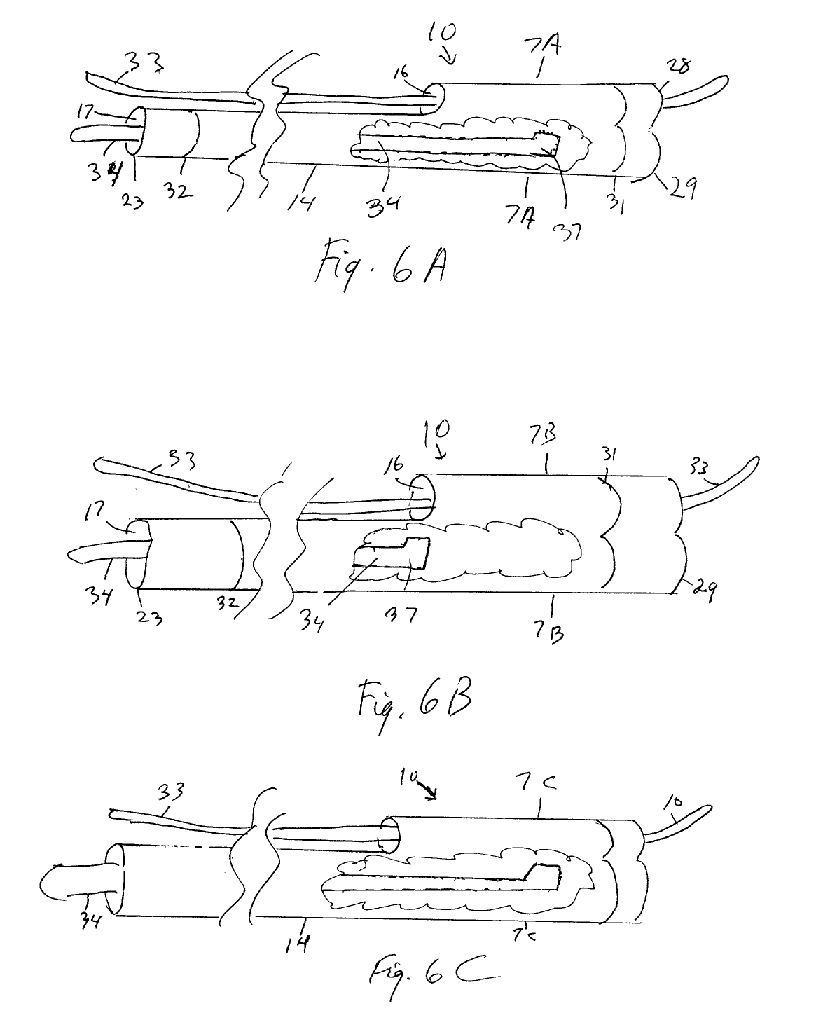

FIGS. 5A, 5B and 5C illustrate a system 10 including an sheath 14 having two lumens 16 and 17, and an intracorporeal imaging instrument 34 having inflatable balloons 61. A guidewire 33 is enclosed within lumen 16, and intracorporeal imaging instrument 34 is enclosed within lumen 17. The balloons 61 are effective to stabilize imaging instrument 34 within lumen 17 of sheath 14 during image acquisition and to provide a controlled optical environment adjacent the imaging elements 37. Balloons 61 may be inflated with a fluid, such as water or saline, having the same or similar optical properties as the blood within a body lumen such as a blood vessel, effective to minimize possible optical distortion. In addition, contact between the balloons 61 and sheath 14 aids in the maintenance of the position of optical elements 37 within lumen 17 in the event of movement of the body lumen within the patient. FIG. 5A illustrates an embodiment where the guidewire lumen 16 is configured as a RX lumen. FIG. 5B is a longitudinal cross-sectional view of sheath 14, showing an embodiment where the guidewire lumen 16 is configured as an OTW lumen. FIG. 5C is a transverse cross-sectional view of a dual-lumen sheath taken along line 5C-5C in FIGS. 5A and 5B.

FIGS. 5D and 5E illustrate an intracorporeal imaging instrument 34 having optical elements 37 placed distal to balloons 61. In FIG. 5D, the intracorporeal imaging instrument 34 is illustrated in place within sheath 14 within a lumen 17. In FIG. 5E, the intracorporeal imaging instrument 34 is illustrated with optical elements 37 and balloons 61 extending beyond sheath 14. Alternatively, an intracorporeal imaging instrument 34 may be positioned with balloons 61 within a sheath 14 and with optical elements 37 extending beyond sheath 14. Portions of FIGS. 5A-5E are shown in longitudinal cross-section.

Insertion of an instrument into a body lumen requires care that the instrument not damage body tissue and so cause harm to a patient. Particularly where the lumen is small, does not follow a straight path, or is branched, as with a cardiovascular lumen, care must be taken when introducing and advancing an instrument into the lumen. For example, where a cardiovascular lesion is involved, it is desirable to position an imaging guidewire or imaging catheter across the lesion within an affected blood vessel. After such distal positioning within a lumen across a lesion, where a clinician wishes to observe or document the condition of a body lumen during an invasive procedure, the imaging instrument must be retracted proximally through the vessel or lesion in order to obtain imaging information pertaining to the lesion.

Removal of the instrument is not as difficult or dangerous as insertion, since the exit path is along the entry path occupied by the proximal portions of the instrument. Thus, removal of an instrument from a desired location within a body lumen, including partial removal, is typically much easier, simpler, and safer to accomplish than is insertion of the instrument. In particular, the removal of a guidewire, catheter, endoscope, or other such invasive instrument is typically much easier than insertion of such instruments.

Since retraction (proximal movement) of an instrument located within a lumen is easier and safer than advancement (distal movement) of the instrument, imaging instruments are preferably moved proximally during an imaging scan of a body lumen. However, such retraction of the instrument requires the subsequent advancement of the instrument in order to regain a position near to its previous position. Such subsequent advancement of the instrument may be useful, for example, when diseased portions of the lumen are discovered by the imaging acquisition, when further distal locations remain to be inspected, or in other situations. A sheath embodying features of the present invention remains in position even after an enclosed instrument has been retracted, effective to guide such an instrument back to a prior distal position. The sheath thus provides a pathway that constrains the motion of instruments contained within it, guiding them along a preferred route and preventing their entry into adjoining body lumens or into undesired lumenal pathways. For example, where an instrument, positioned within a sheath in place within a body lumen, has been retracted from a distal position within the body lumen, a sheath embodying features of the invention constrains motion of such instruments to a desired path as they are advanced back to a former, distal position within the body lumen. By allowing the ready advancement of an intracorporeal imaging instrument following its retraction, thus avoiding the loss of the time and effort expended in delivering the instrument to its initial distal position, the use of the guiding sheath of the present invention makes practical the use of the preferred “pull back” mode of imaging.

The present invention further comprises methods for using the novel sheaths of the invention. Its transparency is useful to allow an intracorporeal imaging instrument to obtain imaging information from within the sheath. An embodiment of the novel methods comprises a method of guiding within a body lumen an intracorporeal imaging instrument. In some embodiments, the imaging instrument is an optical imaging instrument that is sensitive to a wavelength of optical radiation. This method comprises locating a sheath of the invention within a body lumen, where the sheath has a distal end that is translucent to radiation to which the imaging instrument is sensitive. The intracorporeal imaging instrument is located at a distal location within at least a portion of the sheath, and thus at a distal location within a body lumen. The method further includes moving the intracorporeal imaging instrument in a proximal direction within at least a portion of the sheath. A further step may include moving the intracorporeal imaging instrument in a distal direction within at least a portion of the sheath. In another embodiment of this method, the step of moving the intracorporeal imaging instrument in a distal direction within at least a portion of the sheath is effective to return the intracorporeal imaging instrument near to the distal location within the body lumen. In some embodiments of the methods, the intracorporeal imaging instrument is an optical imaging instrument that is sensitive to a wavelength of optical radiation. Where the imaging instrument is an optical imaging instrument sensitive to a wavelength of optical radiation, the sheath is translucent to that wavelength of optical radiation.

A further embodiment of the methods comprises a method of obtaining an image within a body lumen with an intracorporeal imaging instrument. In some embodiments, the intracorporeal imaging instrument is an optical imaging instrument sensitive to a wavelength of optical radiation. This method comprises the steps of locating within a body lumen a sheath of the invention, where the sheath has a distal end that is translucent to radiation to which the imaging instrument is sensitive, locating the intracorporeal imaging instrument within at least a portion of the sheath at a distal location within a body lumen, moving the intracorporeal imaging instrument within at least a portion of the sheath in a proximal direction, and obtaining image information with the intracorporeal imaging instrument. This method may further include returning the intracorporeal imaging instrument to near to the distal location within the body lumen. The returning step may be accomplished by moving the imaging instrument in a distal direction within at least a portion of the sheath.

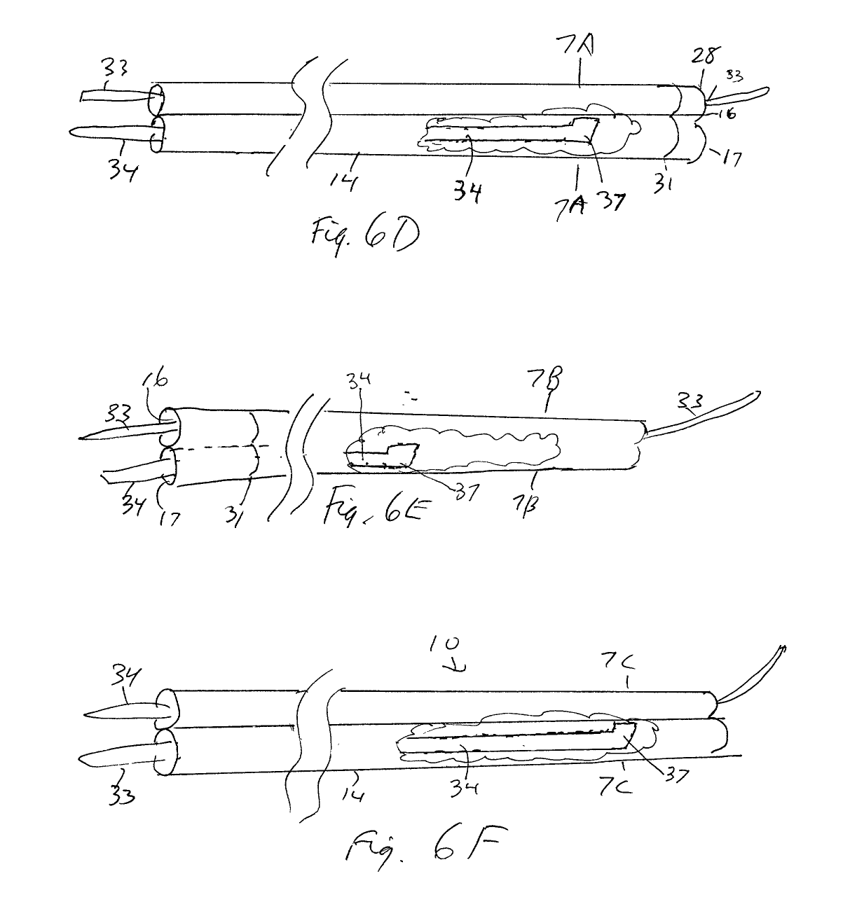

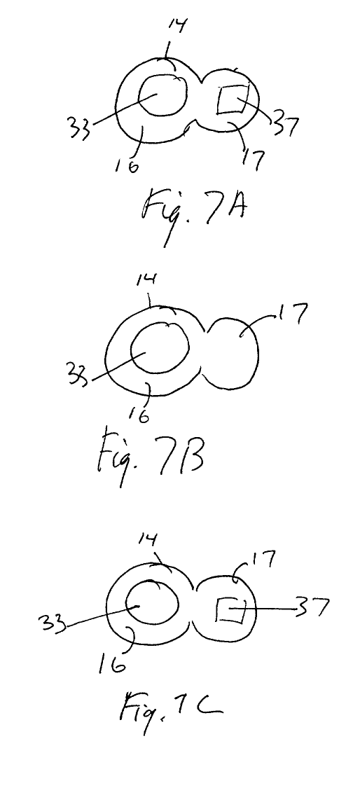

FIGS. 6A-6F and 7A-7C illustrate a method of using an intracorporeal imaging system 10 having a sheath 14 with two lumens. Typically, an image is acquired from within a body lumen by sensing radiation reflected from a patient's body while moving an intracorporeal imaging instrument within a body lumen of a patient. Movement of the intracorporeal imaging instrument during image acquisition, termed “scanning,” is effective to provide image information from an extended region of the patient's body. An episode of such acquisition is termed a “scan.” Typically, a scan is a pullback scan, in which image information is acquired while an imaging instrument moves from a distal position to a more proximal one.

The methods of the invention are also suitable for use with imaging systems 10 having sheaths with one, three, or other numbers of lumens. In addition, the methods of the invention are suitable for use with sheaths configured as RX or as OTW sheaths. FIGS. 6A-6C are perspective views, including portions showing longitudinal cross-sectional views of sheaths configured as RX sheaths, while FIGS. 6D-6F are perspective views including partial longitudinal cross-sectional views of sheaths configured as OTW sheaths.

The methods of the invention include providing sheaths to aid in the scanning of a body lumen by an intracorporeal imaging instrument. FIGS. 6A-6F and 7A-7C illustrate the methods as practiced with sheaths having two lumens, the lumens being shown in transverse cross section in FIGS. 7A-7C. The sheaths illustrated in FIGS. 6A-6C have a RX configuration, while the sheaths depicted in 6D-6F have OTW configuration. FIGS. 7A-7C illustrate transverse cross-sections taken at a distal location where the cross-sections of RX and OTW sheaths are the same. FIGS. 6A-6F show the position of guidewire 33 and imaging instrument 34 having imaging elements 37 as imaging instrument 34 is first shown in an initial distal position (shown in 6A, 6D and 7A), then retracted, or “pulled back” from that position to a more proximal position within the sheath 14 (shown in FIGS. 6B, 6E and 7B) and finally advanced distally again within lumen 17 back to the initial position, as shown in FIGS. 6C, 6F and 7C. Where sheath 14 is in place within a patient's body, sheath 14 provides the means to return the imaging instrument 34 to its initial position within a patient's body following imaging of a lumen during “pull-back.”

FIG. 7A is a transverse cross-section (taken along line 7A-7A shown in FIG. 6A) of a sheath 14 having a lumen 16 enclosing a guidewire 33 and a lumen 17 enclosing an imaging instrument 34. FIG. 6A is a perspective view of the system 10 as shown in FIG. 7A, where sheath 14 is a RX sheath. FIG. 6D is a perspective view of the system 10 as shown in FIG. 7A, where sheath 14 is an OTW sheath. The sheath 14, guidewire 33 and imaging instrument 34 in FIGS. 6A, 6D and 7A are shown configured for the beginning of a pullback imaging scan in which the imaging instrument 34 is in an initial position within a body lumen, typically with the imaging elements 37 distal of, or adjacent, a suspected lesion within the body lumen. FIGS. 6B, 6E and 7B (a transverse cross-section along line 7B-7B in FIG. 6B) illustrate the system 10 after imaging instrument 34 has been pulled back to a proximal position, typically one proximal of the lesion within the patient's body. FIGS. 6C, 6F and 7C (a transverse cross-section along line 7C-7C in FIG. 6C) illustrate the system 10 after imaging instrument 34 has been returned by distal movement to its previous, initial position.

Imaging information may be collected at any position within the translucent sheath 14, or as imaging instrument 34 is moved from one position to another. It will be understood that such movement, and the collection of image information during or after such movement, may also be in a distal to proximal direction as well as the proximal to distal direction illustrated in FIGS. 6 and 7.

The sheath may be guided by a guidewire to its desired position within a body lumen. Where the intracorporeal imaging instrument is an imaging guidewire, the sheath may be guided to its desired position within a body lumen by the intracorporeal imaging instrument. Once in position, a sheath embodying features of the invention is effective to guide an instrument or instruments enclosed within the sheath, as, for example, when guiding the pullback and subsequent distal replacement of an intracorporeal imaging instrument during and after an imaging scan. Thus, by positioning the distal end of the sheath distal to the lesion, the sheath provides a conduit which easily guides an imaging instrument back into position after scanning a lesion; in addition, multiple proximal and distal movements may be readily performed using the easy guidance of the sheath.

A sheath embodying features of the invention may be used during angioplasty procedures, for imaging and for angioplasty itself. For example, a method of performing angioplasty with an intracorporeal imaging instrument having a balloon includes steps of locating at least a portion of a distal portion of the intracorporeal imaging instrument within at least a portion of the sheath at a distal location within a body lumen, moving the intracorporeal imaging instrument proximally within the sheath, moving the intracorporeal imaging instrument distally within the sheath effective to position the intracorporeal imaging instrument near to a suspected lesion site, and inflating the balloon. The intracorporeal imaging instrument may have imaging elements located distal of a balloon. At least a portion of a balloon may be within the sheath during said inflating step; alternatively, at least a portion of a balloon may be distal of the sheath during the inflating step.

A sheath embodying features of the invention may have smooth surfaces. A smooth exterior surface protects a body lumen from damage during placement and use of a sheath, and makes placement of the sheath easier. A smooth interior surface makes movement of enclosed instruments easier and smoother, providing better images by reducing vibration and oscillation of imaging elements 37 during a scan. In addition, a sheath embodying features of the invention will provide protection to a body lumen from possible damage from rotating elements where an imaging instrument 34 is rotated during a scan.

An intracorporeal optical imaging instrument is an instrument that is sensitive to at least one wavelength of optical radiation configured to be used within a patient's body. Such an imaging instrument may be an imaging guidewire, an imaging catheter, an endoscope, or other device, and may include an optical fiber, a camera, a charge-coupled device, or any other type of instrument suitable for sensing optical radiation effective to provide information useful for forming an image. In forming an image within a body lumen, such as within a blood vessel, optical radiation obtained by an intracorporeal imaging instrument may pass through different materials, including bodily fluids, artificial fluids, and lens or window material of the imaging instrument.

Internal body lumens are typically small; accordingly, a sheath for use within a body lumen will have a small diameter in order to allow it to fit within a body lumen. A well-known property of translucent media is that of refraction; light is refracted as it passes from a medium with one index of refraction into another medium with a different index of refraction. In addition, a small radius of curvature of a refracting material causes greater refraction of transmitted light than does a refracting material with a larger radius of curvature (a gentler curve). This property of translucent media is the basis for optical lenses, and thus this effect of curvature on transmitted light may be termed a “lens effect.”

A sheath enclosing an intracorporeal imaging instrument, because of its small size and thus small radius of curvature, will have a large lens effect which may distort an image obtained from optical radiation passing through the sheath. The present inventors have discovered that the undesirable lens effect of a translucent sheath due to its small radius of curvature can be minimized where the refractive index of the sheath is similar to the refractive index of the surrounding medium. The surrounding medium will typically comprise a bodily fluid such as blood, blood plasma, urine, a gastrointestinal fluid, cerebrospinal fluid, interstitial fluid, or the like, but may include an artificial fluid suitable for introduction into a body lumen. In particular, for a sheath enclosing an intracorporeal imaging instrument for use within a blood vessel, optical distortion due to refraction of optical radiation can be minimized by forming the sheath of materials with an index of refraction similar to the index of refraction of blood, so that there is little if any change in refractive index as optical radiation is transmitted from the blood into the sheath.

Image information is most useful when it has been obtained without a substantial amount of distortion, e.g., when refractive errors are minor. Where there is little or no difference between the indices of refraction of two media, optical radiation passing from one medium to another will undergo substantially no refraction, and an image formed by the optical radiation will thus undergo substantially no distortion.

The index of refraction of a material is a measure of the ability of the material to refract optical radiation. Optical radiation traveling through a first material along a path incident to the surface of a second material is refracted to travel along a different path as it travels through the second material. The angle of the incident path and of the refracted path with respect to the surface of the second material are used to calculate the index of refraction, which is equal to the ratio of the sine of the angle of incidence to the sine of the angle of refraction. The indices of refraction of most common materials, including biological materials such as blood plasma, urine, and other bodily fluids are known and may be found in standard reference books and reference sources. For example, the index of refraction of blood plasma (1 g/100 g solution) is about 1.34 (see, e.g., CRC Handbook of Chemistry and Physics (52nd Edition) page D-210).

Fabrication of guiding sheaths of the invention from materials comprising an index of refraction similar to that of a bodily fluid such as blood plasma, urine, or other bodily fluid or an artificial fluid suitable for introduction into a body lumen, provides a sheath that causes a minimal amount of refraction to optical radiation passing through the sheath to or from a fluid. An index of refraction of a material is similar to the index of refraction of a fluid if the index of refraction of the material is within about 10%, preferably within about 5%, of the index of refraction of the fluid. Fabrication of the sheath from materials having an index of refraction of between about 1.2 to about 1.5 minimizes or eliminates possible distortion of image information due to refraction of optical radiation as it traverses the sheath. In some embodiments of the invention, sheaths embodying features of the invention may include materials having indices of refraction of between about 1.3 to about 1.4. In further embodiments of the invention, sheaths embodying features of the invention may include materials having indices of refraction of about 1.34.

Accordingly, in some embodiments of the present invention, the novel sheath comprises a material with an index of refraction that is substantially the same as the index of refraction of blood plasma and clinical saline solution, that is, about 1.34. In such embodiments, an optical imaging instrument will be able to obtain imaging information from within a novel sheath of the invention with substantially no distortion.

The sheath is translucent to an optical wavelength to which the optical imaging instrument is sensitive. Translucent materials suitable for use in medical devices with indices of refraction within the range desired for a sheath of the invention comprise fluorocarbon polymers such as fluorinated ethylene propylene (FEP), polytetrafluorethylene (Teflon®), perfluoroalkoxy polymers (PFA), ethylene tetrafluoroethylene copolymers (ETFE) and blends thereof. FEP is one suitable material having an index of refraction that is nearly identical to that of blood, blood plasma, urine and of clinically-used saline solutions.

It is often advantageous for an intracorporeal instrument to carry radiopaque markings to facilitate the determination of its position within a patient's body. The sheath may comprise a radiopaque marker, or a plurality of radiopaque markers. Radiopaque markers may comprise gold, tungsten, silver, platinum, alloys and mixtures of these metals, or other biocompatible radiopaque material. A radiopaque marker or markers may comprise bands, bars, dots, a mesh, or other shape or configuration compatible with incorporation into or placement on an intracorporeal sheath.

A sheath of the invention may comprise a tubular construction with a diameter of between about 0.5 mm and about 15 mm. In some embodiments of the invention, the sheath may have a diameter of between about 1 mm and about 10 mm. In alternative embodiments, sheaths embodying features of the invention have lumens configured to accommodate guidewires, catheters, and other intracorporeal instruments having outer diameters of about 0.014″ or having outer diameters of about 0.018″. In yet further embodiments, as, for example, where a sheath embodying features of the invention is used to enclose an intravascular ultrasound imaging instrument, the sheath may be configured to enclose an instrument having an outer diameter of about 0.04″.

In addition, a sheath for use with clinical optical imaging instrumentation must be suitable for sterilization. Radiation sterilization, including electron-beam sterilization, is a common method of sterilization. Polymeric materials are suitable for electron-beam sterilization. FEP is a suitable material that is more resistant to degradation by electron-beam sterilization than other polymeric materials.

While particular forms of the invention have been illustrated and described, it will be apparent that various modifications can be made without departing from the spirit and scope of the invention. Accordingly, it is not intended that the invention be limited, except as by the appended claims.

What is claimed is:

1. A sheath for use with an intracorporeal imaging instrument, comprising a lumen having an inner surface, a longitudinal axis, and a distal portion, said distal portion being translucent to radiation, said sheath being configured to enclose at least a portion of an intracorporeal imaging instrument, said inner surface being configured to slidingly engage said intracorporeal imaging instrument effective to guide longitudinal movement of said intracorporeal imaging instrument within said lumen.

2. The sheath of claim 1, wherein said radiation comprises ultrasound radiation.

3. The sheath of claim 1, wherein said radiation comprises optical radiation.

4. The sheath of claim 1, where said sheath comprises a polymer.

5. The sheath of claim 1, further comprising a radiopaque marker.

6. The sheath of claim 3, wherein said optical radiation comprises optical radiation having a wavelength of between about 0.1 micron to about 3 micron.

7. The sheath of claim 3, wherein said optical radiation comprises optical radiation having a wavelength of between about 0.75 micron to about 2.5 micron.

8. The sheath of claim 3, wherein said sheath comprises a material with an index of refraction of between about 1.2 and about 1.5.

9. The sheath of claim 8, wherein said sheath comprises a material with an index of refraction of between about 1.3 and about 1.4.

10. The sheath of claim 3, wherein the sheath comprises a material selected from the group consisting of fluorinated ethylene propylene (FEP), polytetrafluorethylene (Teflon®), perfluoroalkoxy polymers (PFA), ethylene tetrafluoroethylene copolymers (ETFE), and blends thereof.

11. The sheath of claim 10, wherein the sheath comprises FEP.

12. The sheath of claim 1, wherein said sheath comprises an over-the-wire design.

13. The sheath of claim 1, wherein said sheath comprises a rapid exchange design.

14. A system comprising an intracorporeal imaging instrument and a sheath for use with an intracorporeal imaging instrument, said sheath comprising a lumen having an inner surface, a longitudinal axis, and a distal portion, said distal portion being translucent to radiation, said sheath being configured to enclose at least a portion of an intracorporeal imaging instrument, said inner surface being configured to slidingly engage said intracorporeal imaging instrument effective to guide longitudinal movement of said intracorporeal imaging instrument within said lumen.

15. The system of claim 14, wherein said intracorporeal imaging instrument comprises an optical imaging instrument sensitive to a wavelength of optical radiation.

16. The system of claim 15, wherein said wavelength of optical radiation comprises a wavelength of between about 0.1 micron to about 3 micron.

17. The system of claim 15, wherein said wavelength of optical radiation comprises a wavelength of between about 0.75 micron to about 2.5 micron.

18. The system of claim 14, wherein said intracorporeal imaging instrument comprises an ultrasound imaging instrument.

19. The system of claim 14, wherein said sheath comprises a material with an index of refraction of between about 1.2 and about 1.5.

20. The system of claim 19, wherein said sheath comprises a material with an index of refraction of between about 1.3 and about 1.4.

21. A method of guiding within a body lumen an intracorporeal imaging instrument having a distal portion, comprising:
locating within a body lumen a sheath having a distal portion and a proximal portion, said portions defining a distal direction and a proximal direction, said distal portion being translucent to radiation,
locating at least a portion of said distal portion of said intracorporeal imaging instrument within at least a portion of said sheath at a distal location within said body lumen,
moving said portion of said intracorporeal imaging instrument within said at least a portion of said sheath in a proximal direction, and
moving the portion of the intracorporeal imaging instrument within said portion of said sheath in a distal direction effective to position said intracorporeal imaging instrument near to said distal location.

22. The method of claim 21, further comprising a step of sensing radiation that passes through said translucent distal portion effective to obtain imaging information.

23. The method of claim 22, wherein said intracorporeal imaging instrument comprises a balloon, further comprising a step of inflating a balloon.

24. A method of obtaining an image within a body lumen with an intracorporeal optical imaging instrument sensitive to a wavelength of optical radiation, said intracorporeal optical imaging instrument having a distal portion, comprising:
locating within a body lumen a sheath having a distal end that is translucent to said wavelength of optical radiation and a proximal end, said distal end defining a distal direction and said proximal end defining a proximal direction,
locating at least said distal portion of said intracorporeal optical imaging instrument within at least a portion of said sheath at a distal location within said body lumen,
moving said intracorporeal optical imaging instrument within said at least a portion of said sheath in a proximal direction, and
sensing optical radiation with said intracorporeal optical imaging instrument effective to obtain imaging information.

25. The method of claim 24, further comprising moving the intracorporeal imaging instrument within said sheath near to said distal location within the body lumen.

26. The method of claim 25, wherein the step of moving the intracorporeal imaging instrument near to said location within the body lumen comprises moving the imaging instrument in a distal direction effective to position said intracorporeal imaging instrument within at least a portion of the sheath.

27. The method of claim 24, wherein said intracorporeal imaging instrument has a balloon, further comprising inflating a balloon.

28. A device for use within a body lumen containing a fluid, said fluid having an index of refraction, said device comprising a sheath having an index of refraction similar to said index of refraction of said fluid.

29. The device of claim 28, wherein said sheath is configured to enclose an imaging catheter having a balloon.

30. The device of claim 29, wherein said device is configured to guide an imaging catheter having a balloon to a position within a body lumen for performance of balloon angioplasty.

31. A method of performing angioplasty within a body lumen, using an intracorporeal imaging instrument having a distal portion and a balloon, comprising:
locating within a body lumen a sheath having a distal portion and a proximal portion, said portions defining a distal direction and a proximal direction, said distal portion being translucent to radiation,
locating at least a portion of said distal portion of said intracorporeal imaging instrument within at least a portion of said sheath at a distal location within said body lumen,
moving said portion of said intracorporeal imaging instrument within said at least a portion of said sheath in a proximal direction,
moving the portion of the intracorporeal imaging instrument within said portion of said sheath in a distal direction effective to position said intracorporeal imaging instrument near to said distal location, and
inflating said balloon.

32. The method of claim 31, wherein said intracorporeal imaging instrument comprises imaging elements located distal of said balloon.

33. The method of claim 31, wherein said balloon has portions, and wherein at least a portion of the balloon is within said sheath during said inflating step.

34. The method of claim 31, wherein said balloon has portions, and wherein at least a portion of the balloon is distal of said sheath during said inflating step.
